(19) United States
(12) Patent Application Publication
(10) Pub. No.: US 2016/0074586 A1
(43) Pub. Date: Mar. 17, 2016

(54) AN INJECTION DEVICE

(71) Applicant: INJECTOR ApS, København Ø (DK)

(72) Inventors: Morten Mernøe, Cøpenhagen Ø (DK); Daniel Bar-Shalom, Kokkedal (DK)

(73) Assignee: INJECTOR ApS, København Ø (DK)

(21) Appl. No.: 14/888,544

(22) PCT Filed: May 2, 2014
(86) PCT No.: PCT/DK2014/050120

§ 371 Date: Nov. 2, 2015

(30) Foreign Application Priority Data
May 2, 2013 (DK) PA 2013 70247

(51) Int. Cl. A61M 5/28; A61M 5/32; A61M 5/24

(52) U.S. Cl. CPC A61M 5/284; A61M 5/2448; A61M 5/3294

(57) ABSTRACT

Disclosed herein is a ready-to-use injection device for single use and a container for such device. The container comprises at least one first closed chamber for containine an active substance and optionally a second closed chamber for containing a quid substance comprising an active substance or a solvent. The two chambers are separated by a separation wall which is impermeable and breakable upon exertion of an external force, resulting in enhanced mixing of the substances contained in the device with a minimal formation of air bubbles. The present injection device is safe and particularly well suited for enhanced mixing of pharmaceutically active compositions to be injected in patients and can be used for vaccination programs.

### FIELD OF INVENTION

The invention relates to a container for a safe injection device. The container comprises at least one first closed chamber for containing an active substance and optionally a second closed chamber for containing a liquid substance comprising an active substance or a solvent. The two chambers are separated by a separation wall which can be broken by exertion of an external pressure on the external wall of the second chamber, resulting in mixing of the substances contained in the two chambers. The container can be connected to a needle for injecting a liquid composition. The injection device comprising said container is ready to use and cannot be reused.

### BACKGROUND OF THE INVENTION

Injections are the most common health care procedure worldwide. In developing and transitional countries alone, some 16 thousand million injections are administered each year. More than 90% injections are given for therapeutic purposes while 5 to 10% are given for preventive services, including immunization and family planning.

Unsafe injections place patients at risk of disability and death. Reuse of injection devices without sterilization is of particular concern according to the World Health Organization as it may transmit hepatitis B virus (HBV), hepatitis C virus (HCV) and human immunodeficiency virus (HIV), accounting for 32%, 40% and 5% of new infections in 2000, respectively. In addition, inappropriate and unhygienic use of multi-dose vials may transmit bloodborne pathogens. Moreover, an estimated 4.4% of HIV infections and 39% of HBV and HCV infections are attributed to occupational injury. Among the factors which can cause risks in giving injections or collecting blood are the reuse of injection equipment and the use of non-sterile or reprocessed syringes and needles. (World Health Organization: WHO best practices for injections and related procedures toolkit, 2010).

Accordingly, there is a need for safe injection devices. As the WHO states, “syringes with a reuse prevention feature offer the highest level of safety for injection recipients.” The WHO also recommends using a single-dose vial for each patient, in order to reduce cross-contamination between patients.

A preferred injection device would thus be one that contains a single bolus, is ready to use in order to avoid contamination upon bolus preparation in environments where sterility can be hard to achieve, and cannot be reused. At the same time, shelf-life of the medication to be administered should be as high as possible. Several disposable, ready-to-use injection devices have been disclosed. However, none of these devices have solved the problem.

The present invention is directed to a container for an injection device, which overcomes the problems recited above.

### SUMMARY OF THE INVENTION

It is an object of the present invention to provide a ready-to-use, disposable container comprising:
i) at least a first closed chamber for containing an active substance, said first chamber being defined at least partially by said first outer wall;
ii) optionally a second closed chamber for containing a liquid substance comprising an active compound or a solvent, said second closed chamber being defined at least partially by said second outer wall;
wherein:
said active substance contained in the first closed chamber is in a liquid or in a solid form; and/or
said walls are arranged on each side of a middle plane; and/or
said first and second closed chambers are separated by a separation wall for separating the two closed chambers, said separation wall being impermeable and breakable upon exertion of an external force, wherein the elongation at break of the separation wall is less than the smallest of the elongations at break of the first and second outer walls; and/or
said second outer wall is adapted to be pressed towards said first outer wall for dispensing the active substance.

It is a further object of the present invention to provide a ready-to-use, disposable container comprising:
a first closed chamber for containing:
   i) an active substance in a liquid form; or
   ii) an active substance in a solid form;
   said first chamber being defined partially by a first outer wall; and
a second closed chamber for containing a liquid substance comprising an active compound or a solvent, said second chamber being defined partially by a second outer wall; and
a separation wall for separating the two chambers, said separation wall being impermeable and breakable upon exertion of an external force,
   wherein:
the elongation at break of the separation wall is less than the smallest of the elongations at break of the first and second outer walls; and/or
at least one of the first and second outer walls comprises means for facilitating breaking of said separation wall; and/or
the pressure within the second chamber is greater than the pressure within the first chamber.

It is another object of the present invention to provide an injection device comprising such a container connected to a needle, for injection of a liquid composition.

The present injection device can be activated without a plunger. The container of the present invention is particularly well suited for mixing two substances with only limited action required from the user, who only needs to exert a transient pressure on the side of the second chamber in order to allow mixing of the substances contained in the two chambers. The present container allows mixing in such a manner that formation of air bubbles upon mixing and prior to injection is greatly reduced in preferred embodiments. Such a container can prolong storage times by allowing individual storage of compounds which may have a longer shelf-life than the liquid composition to be achieved.

The invention further relates to a disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable within said housing between said first distal position, an intermediate position, and said first proximal position;
a medicine chamber arranged between said pressing surface and said proximal skin contact wall and axially displaceable together with said surfaces, said medicine chamber being compressible in a non-axial direction by displacing said plunger body towards said proximal end;
said needle being attached to said medicine chamber and being arranged for axial displacement between a third distal position and a third proximal position;
injection displacement means, preferably biasing means, adapted to displace said plunger body from said first distal position to said first proximal position such that said plunger body, said medicine chamber and said needle are displaced in the following sequence:
   said plunger body from said first distal position to said intermediate position, and said needle from said third distal position to said third proximal position, wherein said proximal end of said needle projects distally outside said skin contact wall, and
   said plunger body to said first proximal position, wherein said medicine chamber has been compressed such that at least a portion of the liquid therein has been pressed out through said proximal end of said needle,
   said needle from said third proximal position to said third distal position.

In the present context, the term ‘feeding unit’ is synonymous with ‘injection displacement means’.

Definitions

Active Substance

As understood herein, an active substance is any substance or compound, such as a molecule, a macromolecule, a protein, an antibody, a vaccine, a nucleic acid molecule, an antigen, a lipid, or any compound which has an activity on the body, its metabolism, or its immune response.

Breakable Upon Exertion of an External Force

The term “breakable upon exertion of an external force” as used herein in reference to the wall separating the two chambers of the container is to be understood as follows. Said separation wall is such that it can be deformable, but not breakable when the container is in an inactive state, that is when no external force is exerted on any of the external walls of any of the chambers. At the same time, said separation wall is breakable: exerting an external force on the external wall of the second chamber results in an increased pressure in the second chamber, which in turn results in increased forces being exerted on the separation wall, so that said separation wall is broken, ruptured, torn or punctured. Rupturing or breaking of the separation wall allows fluid communication between the two chambers of the container. The separation wall is not restorable once broken, i.e. breaking or rupturing of the separation wall is not reversible.

Chamber

A chamber as understood herein is a compartment which is closed and can contain a liquid or solid substance.

Deformable

The term “deformable” as used herein refers to the ability of a wall to change shape without breaking. The external wall of the first chamber will be hereinafter referred to as the first outer wall; the external wall of the second chamber will be hereinafter referred to as the second outer wall. The separation wall may be deformable so that its shape can be altered by the internal pressures of the chambers on each side of the wall, but the wall only breaks if an external force is applied to the second outer wall, which normally results in an increased internal pressure within the second chamber. The first and second outer walls may be deformable so that their shape can be altered by an external force, such as an external lateral force, but the walls do not break upon application of the said external force.

Distal

The term distal refers herein to the physical location of a part of the injector. A part of the injector is located distally when it is in the half of the injector opposite to the skin contacting surface.

Elongation at Break

Elongation at break, also known as fracture strain, is the ratio between length after breakage and initial length. It expresses the capability of a material to resist changes of shape without crack formation. The elongation at break is determined by tensile testing. Elongation at break is expressed as a percentage.

Impermeable/Water-Tight

The separation wall of the container described herein is impermeable or water-tight, i.e. it does not allow communication or exchange of the substances contained in the two chambers surrounding said separation wall, unless it is broken.

Needle

A needle as understood herein refers to a needle for injecting a liquid composition, such as a pharmaceutical composition. Such a needle may have zero, one or two sharp ends In the case of needles with two sharp ends, the sharp distal end can be used to rupture a breakable seal such as a membrane which is comprised in the means for attaching a needle, for example on the wall of the second chamber, so that the composition contained in the container can flow within the needle. The needle can be capped at the proximal end, to avoid accidental sticking or spillage of the liquid composition of the container. The means for attaching a needle comprise conventional means known in the art: the needle may be glued to the container, encased in the container, or the means for attaching a needle is such as a luer lock.

Pressure

Pressure is the effect of a force applied to a surface. Pressure is the amount of force acting per unit area. The symbol of pressure is p. The internal pressures herein referred to relate to the pressures within the chambers of the container. The chambers may be filled and/or sealed under conditions where the pressure is lower than the atmospheric pressure, so that their internal pressure is inferior to 1 atm (or 1.0197×10⁻⁵ Pa).

Proximal

The term proximal refers herein to the physical location of a part of the injector. A part of the injector is located distally when it is in the same half of the injector as the skin contacting surface.

Retraction

The term retraction is herein used in reference to a movement of the plunger from a proximal position where it is located within the housing of the injector to a distal position, where it is located outside the housing of the injector. The plunger is retracted when the user exerts a pulling force on it.

Separation Wall

The separation wall is the wall that is in contact with the two chambers of the container and prevents the substances contained therein to flow together. The separation wall of the present container is impermeable and breakable upon exertion of an external force, and normally also deformable in that it can resist the internal pressures of the chambers surrounding it. Thus, when the container is in an inactive state, the separation wall prevents any exchange between the substances of the two chambers.

It is an object of the present invention to provide a ready-to-use, disposable container comprising:
i) at least a first closed chamber for containing an active substance, said first chamber being defined at least partially by said first outer wall;
ii) optionally a second closed chamber for containing a liquid substance comprising an active compound or a solvent, said second closed chamber being defined at least partially by said second outer wall;
wherein:
said active substance contained in the first closed chamber is in a liquid or in a solid form; and/or
said walls are arranged on each side of a middle plane; and/or
said first and second closed chambers are separated by a separation wall for separating the two closed chambers, said separation wall being impermeable and breakable upon exertion of an external force, wherein the elongation at break of the separation wall is less than the smallest of the elongations at break of the first and second outer walls; and/or
said second outer wall is adapted to be pressed towards said first outer wall for dispensing the active substance.

It is a further object of the present invention to provide a ready-to-use, disposable container comprising:
a first closed chamber for containing:
   i) an active substance in a liquid form; or
   ii) an active substance in a solid form;
   said first chamber being defined partially by a first outer wall; and
a second closed chamber for containing a liquid substance comprising an active compound or a solvent, said second chamber being defined partially by a second outer wall; and
a separation wall for separating the two chambers, said separation wall being impermeable and breakable upon exertion of an external force,
   wherein:
the elongation at break of the separation wall is less than the smallest of the elongations at break of the first and second outer walls; and/or
at least one of the first and second outer walls comprises means for facilitating breaking of said separation wall; and/or
the pressure within the second chamber is greater than the pressure within the first chamber.

The active substance contained in the at least one first chamber can be a pharmaceutically active substance, such as a vaccine or a medical formulation. When the container does not comprise a second chamber, the active substance contained in the first chamber is preferably in a liquid form.

In some embodiments, the container comprises only one first chamber containing an active substance. The active substance is then preferably in a liquid form. In such embodiments, the dimensions of the second outer wall in relation to the dimensions of the first outer wall are such that the second outer wall can be pressed towards the first outer wall. In other words, the second outer wall is such that when a force is exerted upon it, it is pressed towards the first outer wall, thereby reducing the volume of the first chamber so that it is emptied and the active substance is dispensed.

In some embodiments the container optionally comprises a second chamber, the liquid substance contained in the second chamber can be a solvent or an active compound, such as an adjuvant in solution, so that a liquid composition generated by mixing the substances contained in the first and second chambers is an active solution, such as a pharmaceutically active solution, such as a vaccine. The container disclosed herein thus may allow separate storage of two active substances or compounds, and may thereby increase shelf-life of the container as compared to a container containing a solution consisting of a mixture of the same two substances.

Also provided is a container in which the first chamber contains an active substance in a solid form. In one embodiment the active substance contained in the first chamber is lyophilised; the active substance can be introduced in the first chamber before being lyophilised. In another embodiment the active substance contained in the first chamber is a solution.

The first and second chambers are defined partly by a first and second outer wall, respectively, and separated by a separation wall, which further defines the first and second chambers. Thus in such embodiments the first chamber is fully defined by the first outer wall and the separation wall. The second chamber is fully defined by the second outer wall and the separation wall. The first and second outer walls and the separation walls are impermeable or water-tight so that the substances contained in the first and second chambers are confined in their respective chamber prior to use (in the “inactive” state) and cannot flow from one chamber to the other. In one embodiment, the separation wall is manufactured from a polymer, such as cyclic olefin copolymer or cyclic olefin polymer. The separation wall can be rendered impermeable or liquid-tight by treatment with paraffin or petroleum jelly, or any treatment used in the art to render polymers liquid-tight. The separation wall is preferably breakable, i.e. exertion of an external force in the form of a lateral pressure exerted by the user on the external wall of the second chamber or pushing a needle against the separation wall preferably results in breaking, tearing, puncturing (or a combination thereof) of the separation wall. The container with an intact separation wall is in the “inactive” state, while the container with a ruptured separation wall, in which mixing of the substances contained in the first and second chambers is possible, is in an “active” state. As can be appreciated, the lateral force that the user needs to exert on the external wall of the second chamber preferably only requires the use of one hand.

The first chamber forms a cavity. The dimensions and/or shape of the second chamber may be such that the second chamber can be pushed towards the first chamber, e.g. when the user exerts a lateral force on one or both of the first and the second outer walls with his fingers. When present, the second chamber is of such shape and/or dimensions relative to the first chamber that the second chamber can be at least partly contained within the cavity formed by the first chamber. The second outer wall is adapted to be pressed towards the first outer wall for dispensing the contents of the first chamber. Thus the volume defined by the middle plane and the second outer wall is preferably smaller than the volume defined by the middle plane and the first outer wall.

The first and the second outer walls may be deformable or non deformable upon exertion of a pressure such as a lateral pressure. Preferably at least one of the first and second outer walls is deformable upon pressure. In some embodiments the first outer wall is rigid and not deformable when an external force is exerted upon it; for example, a force resulting from a user applying his fingers on the container when using the container does not result in substantial deformation of the first outer wall. In some embodiments the second outer wall is deformable. Thus in some embodiments application of pressure on the container results in deformation of the second outer wall but not in deformation of the first outer wall.

It will be appreciated that the deformability of the outer walls may be related to the nature of the materials from which said walls are manufactured. Thus in some embodiments, the first outer wall is manufactured from a rigid material such as metal or glass. A non-limiting example of a suitable metal is steel. In such embodiments the first outer wall is not deformable. In other embodiments, the first outer wall is manufactured from a polymer. Examples of suitable polymers include, but are not limited to, cyclic olefin polymer and cyclic olefin copolymer. In particular embodiments, the first outer wall is manufactured from a material which will allow the first outer wall to change shape without breaking when an external force is exerted upon it, such as when a user applies pressure on the walls with his fingers.

In some embodiments, the second outer wall is manufactured from a polymer. Suitable polymers are cyclic olefin copolymer and cyclic olefin polymer. Thus in some embodiments the second outer wall is deformable and is manufactured from a material which will allow the first outer wall to change shape without breaking when an external force is exerted upon it, such as when a user applies pressure on the wall with his fingers.

Thus the first outer wall may be manufactured from metal and the second outer wall from a polymer. In another embodiment, the first outer wall may be manufactured from glass and the second outer wall from a polymer. In yet another embodiment, the first outer wall and the second outer wall may be manufactured from the same polymer or from different polymers.

The deformability of the outer walls may be dependent on the thickness of the walls. Thus a force exerted by the fingers of a user on the first and second outer walls may result in deformation of thinner polymer walls, while thicker polymer walls will not be deformed. Likewise, it will be appreciated that outer walls manufactured from glass may break upon exertion of such a force if they are too thin.

In some embodiments, the second outer wall is more deformable than the first outer wall. The first and second outer walls may also be deformable to the same extent. In some containers of the invention the first and second outer walls are manufactured from the same material but have different thicknesses. The second outer wall may be thinner than the first outer wall, whereby the second outer wall may be more deformable or flexible than the first outer wall. The first and second outer walls may also be manufactured from different materials and have identical or different thicknesses. The first and second outer walls may be equally deformable.

The first outer wall may be manufactured from glass and have a thickness of 1 mm or more, such as 1.1 mm or more, such as 1.2 mm or more, such as 1.5 mm or more, such as 2 mm or more. The first outer wall may be manufactured from metal, such as steel, and have a thickness of 0.5 mm or more, such as 0.75 mm or more, such as 1 mm or more, such as 1.25 mm or more, such as 1.5 mm or more. The first outer wall may be manufactured from a polymer and have a thickness of 1 mm or more, such as 1.1 mm or more, such as 1.2 mm or more, such as 1.5 mm or more, such as 2 mm or more. The second outer wall may be manufactured from a polymer and have a thickness of 0.1 mm or more, such as 0.2 mm or more, such as 0.3 mm or more, such as 0.5 mm or more, such as 0.75 mm or more, such as 1 mm or more, such as 1.25 mm or more, such as 1.5 mm or more, such as 2 mm or more.

In some embodiments the ratio between the thickness of the second outer wall and the thickness of the second outer wall is between 1/5 and 1/20, such as between 1/7 and 1/15, such as between 1/8 and 1/12, such as between 1/9 and 1/11. Preferably the ratio is 1/10.

In some embodiments the first and the second outer walls are directly connected. In some embodiments, the first and the second outer wall may be manufactured from a single piece of manufacturing material. This may be advantageous in the embodiments where the first and second outer walls are manufactured from the same material. In other embodiments, the first and second outer walls may be directly connected so as to define a single entity. The first and second outer walls may thus be welded or glued together, so that the volume they define is impermeable and liquid-tight.

Disclosed herein are embodiments in which the thickness of the separation wall is comprised between 10 and 250 μm, such as between 20 and 200 μm, such as between 30 and 150 μm, such as between 40 and 100 μm, such as between 40 and 90 μm, such as between 40 and 80 μm, such as between 40 and 70 μm, such as between 40 and 60 μm, such as 50 μm. In a preferred embodiment, the separation wall is 50 μm thick.

Preferred embodiments are those in which the elongation at break of the separation wall is equal to or less than 15%, such as equal to or less than 14%, such as equal to or less than 13%, such as equal to or less than 12%, such as equal to or less than 11%, such as equal to or less than 10%, such as equal to or less than 9%, such as equal to or less than 8%, such as equal to or less than 7%, such as equal to or less than 6%, such as equal to or less than 5%, such as equal to or less than 4%, such as equal to or less than 3%, such as equal to or less than 2%.

Also envisioned herein are containers in which the volumes of the first and second chambers are such that the sum of these volumes is comprised between 0.1 and 20.0 mL. For example, the sum of the volumes of the first and second chambers is comprised between 0.2 and 18 mL, such as between 0.3 and 16 mL, such as between 0.4 and 14 mL, such as between 0.4 and 12 mL, such as between 0.4 and 10 mL, such as between 0.4 and 8 mL, such as between 0.5 and 6 mL, such as between 0.6 and 4 mL, such as between 0.5 and 2 mL, such as between 0.6 and 1 mL, such as between 0.7 and 0.9 mL, such as 0.8 mL. In a preferred embodiment the sum of the volumes is equal to 0.8 mL.

In preferred embodiments, the elongation at break of the separation wall is at least equal to the greatest of the elongations at break of the first and second outer walls. In embodiments where the elongation at break of the separation wall is equal to the greatest of the elongations at break of the first and second outer walls, the breaking, tearing, puncturing (or a combination thereof) of the separation wall can be facilitated by protuberances present on the internal side of at least one of the outer walls. Upon exertion of an external force on at least one outer wall, said protuberances are contacted with the separation wall and exert a supplementary tension facilitating its breaking, tearing, puncturing (or a combination thereof). The protuberances of these embodiments can be of any kind that will help facilitate the breaking, tearing, puncturing (or a combination thereof) of the separation wall, as will be evident to the skilled person.

In some preferred embodiments, the internal sides of the external walls of the first and/or second chamber carry at least one protuberance in at least one location along different axes, so that when a mechanical force is exerted on the external wall of the second chamber, said at least one protuberance is contacted with the separation wall, facilitating breaking of the separation wall and resulting in the substances of the two chambers being mixed.

In another preferred embodiment, the internal side of the external wall of the second chamber carries at least one protuberance in at least one location, while the internal side of the external wall of the first chamber is devoid of protuberance. In another embodiment, the internal side of the external wall of the second chamber carries one protuberance in one location along an axis Al and the internal side of the external wall of the second chamber carries two protuberances in two locations along axes A2 and A3; A1, A2 and A3 are not identical and can be parallel. In a particular embodiment, A1 is located between A2 and A3.

The container also comprises means for attaching a needle, or any injection system suitable for injecting a sterile composition, such as a Luer Lock system. The needle of some embodiments can be covered with a cap at its proximal end. At least the proximal end of the needle is sharp. In some embodiments, the distal end of the needle is sharp and can be used to perforate the external wall of the chamber to which the means for attaching a needle are attached, to allow communication between the container and the needle so that the liquid composition can be injected. For example, the user can exert an external force, such as push the needle inside the container. In this embodiment, the axis of the needle and the axis of the separation wall are parallel but not identical. In a preferred embodiment, the needle is connected to the first chamber. In other embodiments, a breakable seal such as a membrane separates the distal end of the needle from the chambers, and the membrane is broken prior to injection.

Fluid communication between the container and the needle may be prevented by e.g. the presence of a breakable seal. The breakable seal can be broken upon exertion of a lateral force on at least one of the first and second outer walls. Breaking of the seal may be facilitated by the at least one protuberance present on the second outer wall. In such embodiments the at least one protuberance is located such that upon exertion of a lateral pressure, the protuberance will come in contact and apply pressure on the seal, whereby breakage is attained.

The breakable seal between the needle and the container may be manufactured essentially as described above for the separation wall. Breaking of the seal results in fluid communication between the container and the needle, thereby allowing the composition or solution contained in the container to flow through the needle. Thus exertion of a lateral force on at least one of the first and second outer walls allows emptying of the container.

Disclosed herein is a container in which the substance in the first chamber is in a solid form and the substance in the second chamber is in a liquid form. In a preferred embodiment enhanced mixing of the substances can be achieved if the pressure within the first chamber is lower than the pressure within the second chamber, so that when the separation wall is broken, the differences in pressure facilitates mixing of the two substances. Such a difference in pressure can be achieved by sealing the first chamber containing the substance in a solid form in conditions where the pressure is lower than the atmospheric pressure, while the second chamber containing the liquid substance is sealed under normal atmospheric conditions. In such embodiments the difference in pressure should be such that it does not cause breaking of the separation wall in the inactive state, but that it can break upon activation, i.e. upon exertion of an external force such as piercing by a needle or application of a lateral mechanical force on the second outer wall. In a preferred embodiment, the external force is a lateral mechanical force exerted by the user with his fingers on the second outer wall. In embodiments where the external force is a mechanical force exerted by the user with his fingers on the second outer wall, the first and second external walls are such that the mechanical pressure does not result in breaking of said external walls. The second outer wall is deformable, i.e. it is possible to increase the internal pressure within the second chamber by exerting a mechanical force on said second outer wall as discussed above, so that the mechanical force results in breaking of the separation wall but not in breaking of the first or of the second outer wall. Because of the difference in pressure between the two chambers, mixing of the substances contained in the two chambers of such a container is enhanced and occurs with minimal formation of air bubbles.

A container as described herein may be housed in a housing. The housing is preferably such that it is possible to exert a mechanical force on said housing, allowing for breaking of the separation wall. The housing may comprise a lid, preferably an internal lid, which can help facilitating breaking of the separation wall. Such an embodiment is described in FIGS. 5 to 8 and in the corresponding description.

Also disclosed herein is a container supplied with a lid; examples of such embodiments are shown in FIGS. 4 and 9. The lid may be hinged on to the container, and is preferably such that applying a force on said lid helps facilitating breaking of the separation wall.

Also within the scope of this application is an injection device comprising a container as described for injecting a liquid composition resulting in the mixing of the substances contained in the first and second chambers of said container. In some embodiments the liquid composition is a vaccine composition. In other embodiments, the liquid composition consists of a bolus of a pharmaceutically active composition. The injection device is in an inactive state as long as the separation wall between the first and second chambers is intact, while it goes into an active state upon exertion of an external force resulting in breaking of the separation wall and mixing of the substances of the first and second chambers of the container, yielding an active liquid composition. After the device has reached the active state, it cannot be brought back to an inactive state, because the separation wall cannot be reformed. Thus the device described herein is auto-destructible and cannot be reused.

The single-use, auto-destructible device described herein is as such particularly well-suited for injection of vaccine compositions in humans in developing countries, minimising contamination risks dues to recycling of injection devices. The container described herein is easy to operate and is ready to use, thus minimising manipulation times and as a result minimising occupational injuries or risks of contamination for the staff performing vaccination programmes.

It is a further object of the invention to provide a disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable within said housing between said first distal position, an intermediate position, and said first proximal position;
a medicine chamber arranged between said pressing surface and said proximal skin contact wall and axially displaceable together with said surfaces, said medicine chamber being compressible in a non-axial direction by displacing said plunger body towards said proximal end;
said needle being attached to said medicine chamber and being arranged for axial displacement between a third distal position and a third proximal position;
injection displacement means, preferably biasing means, adapted to displace said plunger body from said first distal position to said first proximal position such that said plunger body, said medicine chamber and said needle are displaced in the following sequence:
   said plunger body from said first distal position to said intermediate position, and said needle from said third distal position to said third proximal position, wherein said proximal end of said needle projects distally outside said skin contact wall, and
   said plunger body to said first proximal position, wherein said medicine chamber has been compressed such that at least a portion of the liquid therein has been pressed out through said proximal end of said needle,
   said needle from said third proximal position to said third distal position.

In the present context, the term ‘feeding unit’ is synonymous with ‘injection displacement means’.

The sequence through which said plunger body, said medicine chamber and said needle are displaced may be performed automatically through the action of the displacement means.

In some embodiments the injector further comprises a needle guide body provided with a passage for receiving and guiding said injection needle.

In a preferred embodiment the needle guide body has an abutment surface facing in a distal direction towards said pressing surface and arranged for axial displacement between a second distal position, a second intermediate position and a second proximal position.

In a further preferred embodiment said injection displacement means, preferably biasing means, is adapted to displace said plunger body from said first distal position to said first proximal position such that said plunger body, said medicine chamber, said guide body and said needle are displaced in the following sequence:
said plunger body and said guide body from said first distal position and said second distal position, respectively, to said intermediate position and said second intermediate position, respectively, and said needle from said third distal position to said third proximal position, wherein said proximal end of said needle projects distally outside said skin contact wall, said plunger body and said guide body from said intermediate position and said second intermediate position, respectively, to said second intermediate position and said second proximal position, respectively, wherein said distal end of said needle has penetrated into said medicine chamber,
said plunger body from said second intermediate position to said first proximal position, wherein said medicine chamber has been compressed such that at least a portion of the liquid therein has been pressed out through said proximal end of said needle, and
said guide body and said needle from said second intermediate position and said third proximal position, respectively, to said second distal position and said third distal position, respectively.

The sequence through which said plunger body, said medicine chamber, said guide body and said needle are displaced may be performed automatically through the action of the displacement means.

It is preferred that the medicine chamber:
further contains a container for containing an injection liquid and having a proximal wall portion penetrable by said sharp distal end of said needle;
is adapted to be reduced in volume so as to compress said container to press said injection liquid into said needle.

In some embodiments, the injection liquid is a pharmaceutically active liquid.

Having a separate container inside the medicine chamber allows separate manufacturing of the container and of the remaining parts of the injector, thus conveniently allowing containers with various contents to be used with the same injector. As a result a variety of injectors may be produced containing different injection liquids.

The invention further relates to a disposable automatic injector for injecting an injection liquid and comprising:
a cylindrical housing with a distal wall and a proximal wall having a proximal surface for abutting the surface of an injection site,
an injection needle having a sharp distal end and a sharp proximal end and arranged axially displaceable in said housing through a passage in said proximal wall
a compressible container for containing an injection liquid and having a proximal wall portion penetrable by said sharp distal end of said needle,
a compression chamber containing said container and adapted to be reduced in volume so as to compress said container to press said injection liquid into said needle, and
displacement means, preferably biasing means, for:
displacing said compression chamber and said needle in the proximal direction towards said proximal wall such that said needle is extended outside said proximal wall,
causing said sharp distal end of said needle to penetrate into said container,
reducing the volume of said compression chamber, and
displacing said needle, said container and said compression chamber in the distal direction away from said proximal wall such that the sharp proximal end of said needle becomes located within said housing.

In some embodiments, the needle guide body is immobile within the housing. In other embodiments, the needle guide body is arranged so that movement of the needle guide body, for example by application of a pressure on its distal abutment surface, results in movement of the needle in the same direction. For example the needle guide body may be connected to the needle.

The medicine chamber may comprise a top and a bottom surface (4a and 4c, respectively, FIG. 1b). At least one of the top and bottom surfaces of the medicine chamber may be connected to the plunger body. In one embodiment, application of a force through the injection displacement means results in a pressure being exerted on at least one of the top and bottom surface of the medicine chamber, thus resulting in compression of the medicine chamber in a non-axial manner. In some embodiments, only one surface is connected to the plunger and the other surface is immobile relative to the housing. In some embodiments, connection of the at least one of the top and bottom surface of the medicine chamber is achieved by at least one hinge. In some embodiments, at least one of the surfaces of the medicine chamber is hinged to the housing so that movement of the plunger from a proximal position to a distal position results in the hinged surface exerting a pressure on the container placed within the medicine chamber (FIG. 11). The top and the bottom surfaces may both be hinged to the housing to allow the container to be compressed from two directions at once. Other means of attaching the at least one of the surfaces of the medicine chamber are means known in the art, such as a spring, of which one end is attached to the plunger and the other to the at least one surface.

The top and bottom surfaces of the medicine chamber may be planar or curved. Preferably, said surfaces are not parallel and their axes intersect at the proximal end of the injector. The top and bottom surfaces should be so arranged relative to each other that they may at least partly overlap, i.e. they should be so arranged that exertion of a pressure on the walls of the housing enables compression of the medicine chamber by the top and bottom surfaces.

In some embodiments, a spring is positioned between the injection needle and the displacement means. When the plunger is retracted, for example when the user pulls the plunger, the spring is tensioned for being released so that the displacement means is moved forward by the spring force. In some embodiments, said spring is made of rubber. In other embodiments, the displacements means comprises a rubber band.

Also disclosed are embodiments in which the container is made of a plastic film. Preferably, the region of the container which is to be penetrated by the sharp distal end of the needle is provided with a sealing film. Upon penetration of the container by the distal end of the needle, the sealing film seals against the side surface of the needle, thus ensuring that no injection liquid is spilled on the sides of the needle. Such sealing film may for example be made of rubber or of polymers as is known in the art.

In some embodiments, the injection liquid is a pharmaceutically active liquid, such as a vaccine.

It is another object of the invention to provide a disposable injector wherein said container for containing an injection liquid consists of:
a first chamber containing a dry matter;
a second chamber containing a liquid;
a peel section separating the first and the second chamber;
   wherein the peel section is breakable.

In preferred embodiments, the first and second chambers are impermeable, so that the dry matter and the liquid they contain may not spill in the medicine chamber in which the container is placed. The peel section is preferably also impermeable, thus contact between the liquid and the dry matter is prevented as long as the plunger is not being retracted. The peel section is breakable, so that upon retraction of the plunger, breakage of the peel section allows contacting of the dry matter of the first chamber with the liquid of the second chamber, resulting in a liquid composition or injection liquid.

In some embodiments, the dry matter comprises an active substance, such as a pharmaceutically active substance, such as a vaccine. The liquid contained in the second chamber may comprise an active substance or a solvent. In some embodiments, the liquid is a solvent, such that it results in an active solution upon mixing with the active substance of the first chamber.

In some embodiments, complete retraction of the plunger results in mixing of the dry matter and the liquid. For example, the plunger may be provided with protuberances or teeth, so that upon retraction the protuberances get in contact with the top of the medicine chamber, thus resulting in vibrations facilitating mixing of the dry matter and the liquid.

Pharmaceutically active substances have a longer storage life when in a dry form. Active substances in dry form also often display higher stability when confronted to uncontrolled environmental conditions such as high or low temperature, temperature variations or pressure variations. Thus embodiments with two separate chambers may be particularly relevant when the injection liquid is a pharmaceutically active substance, as it may extend the shelf life of the injector.

In some embodiments, the proximal skin contact wall of the housing is provided with an antiseptic means intended for being applied to the injection site. Such antiseptic means may be any means known in the art such as commercial antiseptics, for example trichlorophenylmethyliodosalicyl. In embodiments where the antiseptic means is volatile, the skin contact wall may be covered by an air-tight membrane or peel section to prevent its evaporation.

The invention also relates to an injector, wherein a cross-section of the housing is of rectangular shape. The cross-section of the housing may thus be square, rectangular, and have sharp or round edges. Such embodiments present several advantages compared to injectors where a cross-section of the housing is rectangular. The dead volume of the injector, i.e. the volume which is not essential for its proper functioning, is reduced, thus reducing the total volume of the injector as compared to a circular injector containing the same volume of medicine, and thus allowing storage in a lesser space. The rectangular shape allows the user to get a better grip on the housing as it provides flat, lateral surfaces. The housing may also be equipped with grips on its lateral faces in order to further facilitate manipulation.

In some embodiments, the housing is provided with a window, which may allow visualisation of the medicine chamber, in order to allow the user to check for integrity of the medicine bag, or for correct mixing of the contents of the medicine chamber.

### DESCRIPTION OF THE DRAWINGS

The invention will in the following be described in greater detail with reference to the accompanying drawings:

FIG. 1 a schematic representation of how a container wherein the first closed chamber containing a solid substance can be prepared and used.

FIG. 2 a schematic representation of a container wherein both chambers contain a liquid substance.

FIG. 3 schematic representations of containers comprising means for facilitating breaking of the separation wall.

FIG. 4 a schematic representation of a container with a moveable lid.

FIG. 5 a schematic representation of a container adapted for an automatic injection device, before use.

FIG. 6 a schematic representation of the container of FIG. 5, at the time where the separation wall breaks and the substances of the two chambers get mixed.

FIG. 7 a schematic representation of how the needle of the container of FIG. 6 is pushed out of the injection device.

FIG. 8 a schematic representation of how the liquid composition is expelled from the container and how the needle retracts within the container.

FIG. 9 a schematic representation how the liquid composition is expelled from a container with a single chamber equipped with a needle.

FIGS. 10-16 schematic views of a currently preferred embodiment of an injector according to the invention illustrating sequential positions of the various elements of the injector when carrying out an injection.

FIG. 17 detailed view of part of an embodiment.

### DETAILED DESCRIPTION OF THE DRAWINGS

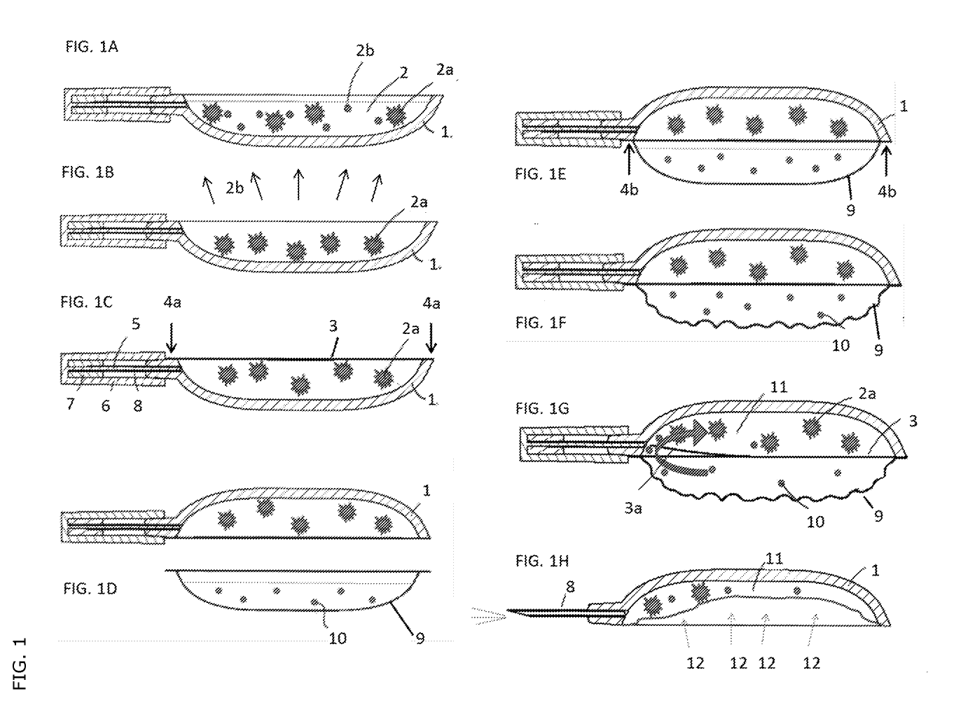

FIG. 1 shows how to prepare and use a container as disclosed herein.

FIG. 1A shows a liquid substance 2, composed of an active substance 2a dissolved in a liquid 2b, the liquid substance 2 being poured in the first chamber being partially defined by a rigid outer wall 1.

FIG. 1B shows the lyophilisation process in which the liquid 2b is evaporated and the active substance 2a is lyophilised.

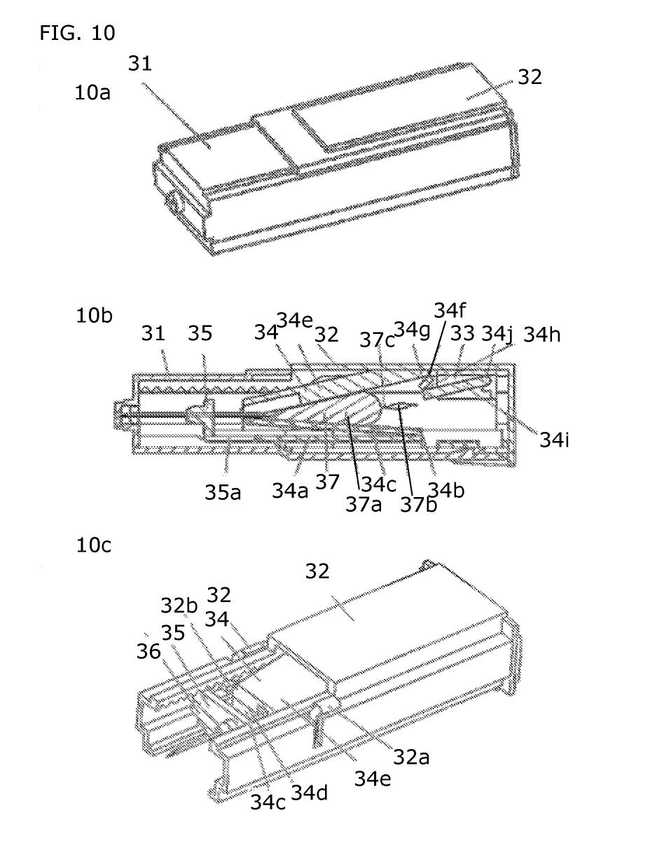

FIG. 10 shows how the separation wall 3 is welded in 4 on to the rigid outer wall 1 of the first chamber. The stylet 5, together with the rubber stopper 7 in the cap 6 ensure that the cannula 8 is closed, so that the first chamber partially defined by the rigid outer wall 1 is now fully defined after welding in 4a of the separation wall 3. If the welding is performed under vacuum the first chamber is filled with lyophilised active substance 2a , and the pressure in the first chamber is lower than the atmospheric pressure.

FIG. 1D shows the first chamber of FIG. 10 turned upside down. The second chamber is partially defined by a flexible, second outer wall 9 and contains a liquid solvent 10.

FIG. 1E shows how the second chamber partially defined by the flexible, second outer wall 9 is welded on to the first chamber delimited by the rigid outer wall 1 by welding in 4b, under vacuum.

FIG. 1F shows the final, assembled container; under atmospheric pressure, the flexible, second outer wall 9 retracts around the liquid solvent 10.

FIG. 1G shows mixing of the lyophilised, active substance 2a contained in the first chamber, and the liquid solvent 10 contained in the second chamber, upon breaking of the separation wall 3. Since the internal pressure within the first chamber containing the lyophilised, active substance 2a is lower than atmospheric pressure, while the liquid solvent 10 in the second chamber is affected by the atmospheric pressure via the flexible, second outer wall 9, the difference in pressure pushes the liquid solvent 10 into the first chamber 1 containing the lyophilised, active substance 2a, whereby the lyophilised, active substance 2a and the liquid solvent 10 are mixed to form a liquid composition 11.

FIG. 1H shows how to use the container. The cap 6, the plug pin 5 and the rubber stopper 7 are removed from the needle 8. A force 12 applied to the flexible, second outer wall 9 expels the liquid composition 11 from the first chamber delimited by the rigid outer wall 1 through the cannula 8.

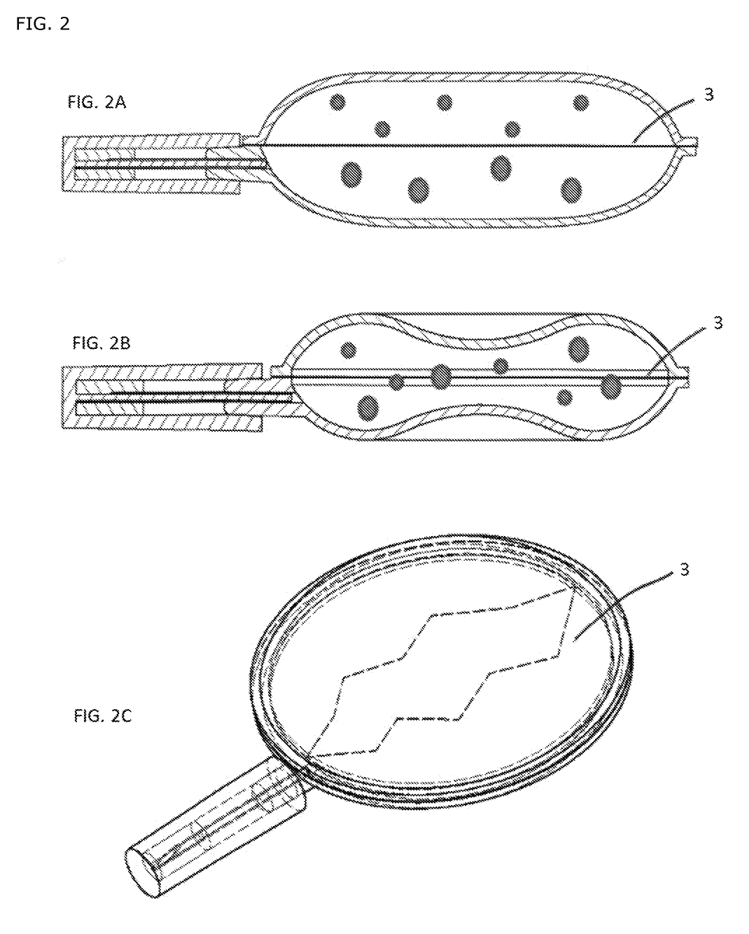

FIG. 2 shows an embodiment in which the first and second closed chambers contain a liquid, and in which vacuum is not needed to achieve a good mixing and avoid air in the resulting liquid composition.

FIG. 2A shows the container before use, with the separation wall 3 intact.

FIG. 2B shows how an external force can be applied on the first and second outer walls, which are both of a flexible material, resulting in breaking of the separation wall 3.

FIG. 2C shows how the separation wall 3 is broken.

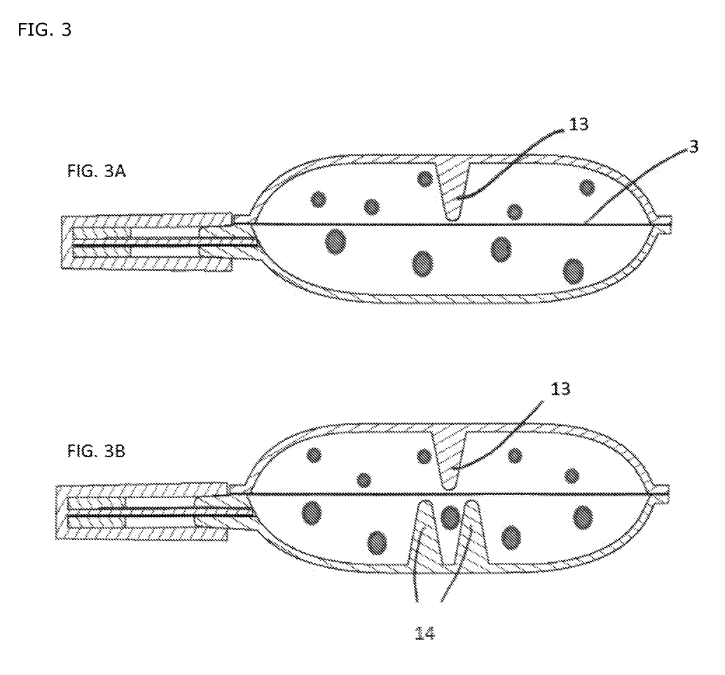

FIG. 3 shows an embodiment similar to the embodiment of FIG. 2, with means for breaking the separation wall 3.

FIG. 3A shows such a container with one protuberance 13 on the inner side of the first outer wall of the first chamber. Upon exertion of an external force, the protuberance 13 is contacted with the separation wall 3, which breaks.

FIG. 3B shows an alternative embodiment with one protuberance 13 on the inner side of the first outer wall of the first chamber, and two protuberances 14 on the inner side of the second outer wall of the second chamber. The protuberances 14 are not aligned with the protuberance 13. Upon exertion of an external force, the protuberance 13 and the protuberances 14 are contacted with the separation wall 3, which breaks.

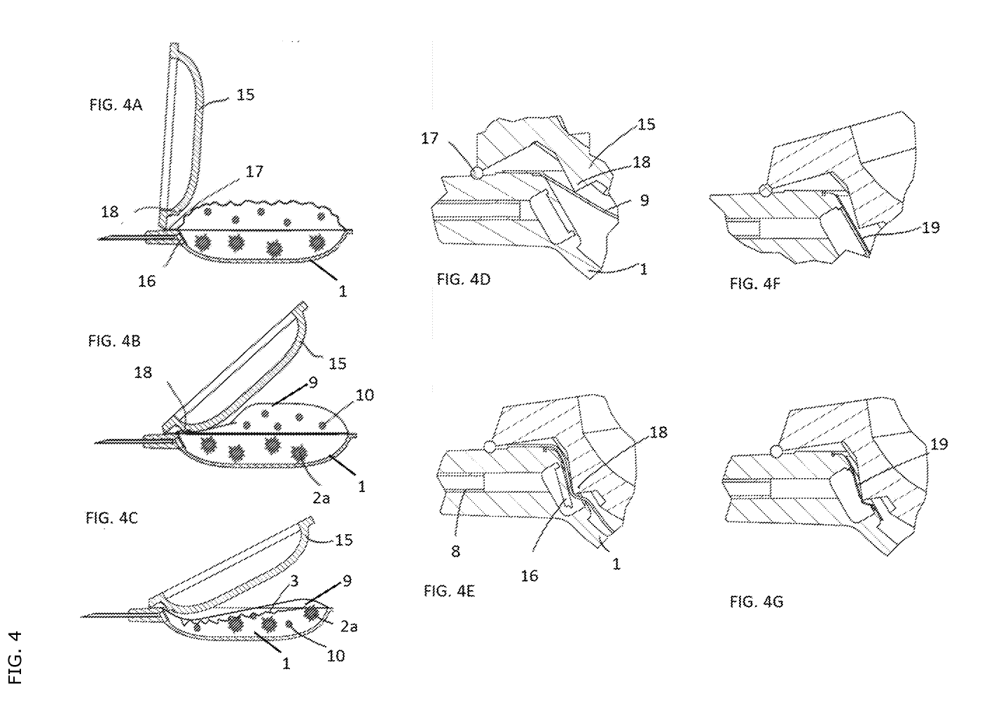

FIG. 4 shows an embodiment similar to the embodiment of FIG. 1, with a moveable lid 15 and a breakable wall 16.

FIG. 4A shows the container ready for use. The lid 15 is hinged in 17 on the rigid wall 1 of the first chamber.

FIG. 4B shows the lid 15 moved partly down into a position where a protuberance 18 on the lid 15 is just getting in contact with the separation wall 3 via the flexible, second outer wall of the second chamber 9, between the liquid solvent 10 and the lyophilised active substance 2a. Since the second outer wall 9 of the second chamber is made of a flexible but resistant material, the second outer wall is not punctured by the protuberance 18 upon contact.

FIG. 4C shows the device with the lid 15 moved further down, the protuberance having just broken the separation wall 3, whereby the atmospheric pressure which is applied to the flexible, second outer wall 9 of the second chamber causes the liquid solvent 10 to flow into the first chamber that was under vacuum delimited by the rigid outer wall 1 containing the lyophilised active substance 2a, whereby the liquid solvent 10 and the lyophilised active substance 2a are mixed to form a liquid composition.

FIG. 4D shows a detailed view of the area surrounding the axis 17 and the protuberance 18, the lid 15 being in the same position as in FIG. 4C.

FIG. 4E shows a detailed view of the lid being moved further down. The protuberance 18 has just broken the breakable wall 16 and the liquid composition to be injected can flow from the first chamber delimited by the rigid outer wall 1 through the cannula 8.

FIG. 4F shows an embodiment similar to the one of FIG. 4D, where the breakable wall is replaced by a breakable membrane 19.

FIG. 4G shows an embodiment similar to the one of FIG. 4E, where the breakable wall is replaced by a breakable membrane 19.

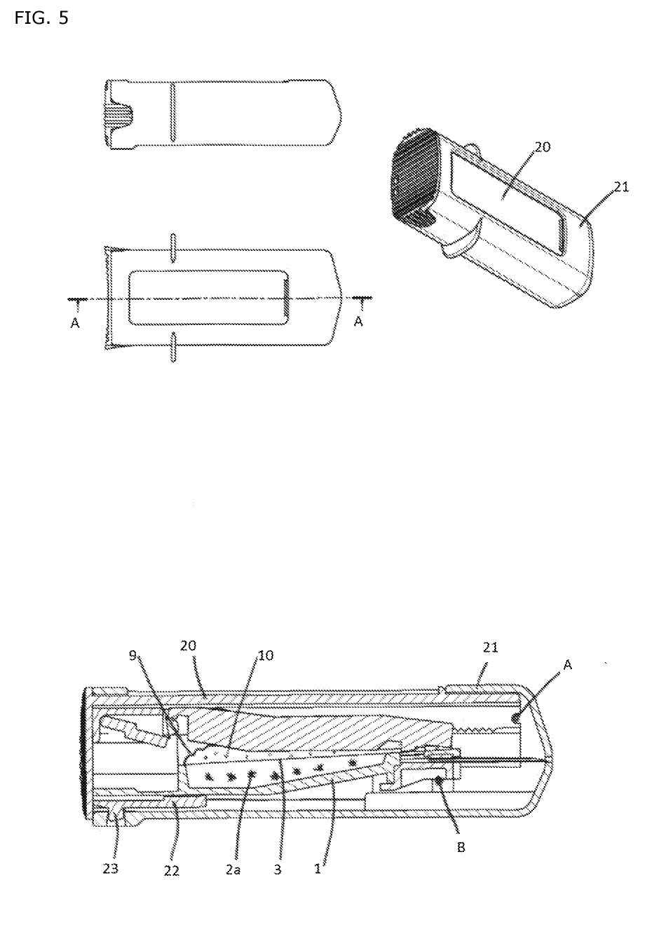

FIG. 5 shows a schematic representation of a container adapted for an automatic injection device, before use. The plunger 20 is pushed completely within the housing 21. The first chamber delimited by the rigid outer wall 1 is guided by the guiding unit 22, which is attached to the housing 21 via the spring catch 23. The first chamber is filled with lyophilised, active substance 2a, and mounted to the separation wall 3 and the second chamber delimited by the flexible wall 9 and filled with liquid solvent 10. The end of a spring (not shown) is attached to point A of the plunger 20. The other end of the spring is attached to point B on the rigid, outer wall 1 of the first chamber. The spring is relaxed.

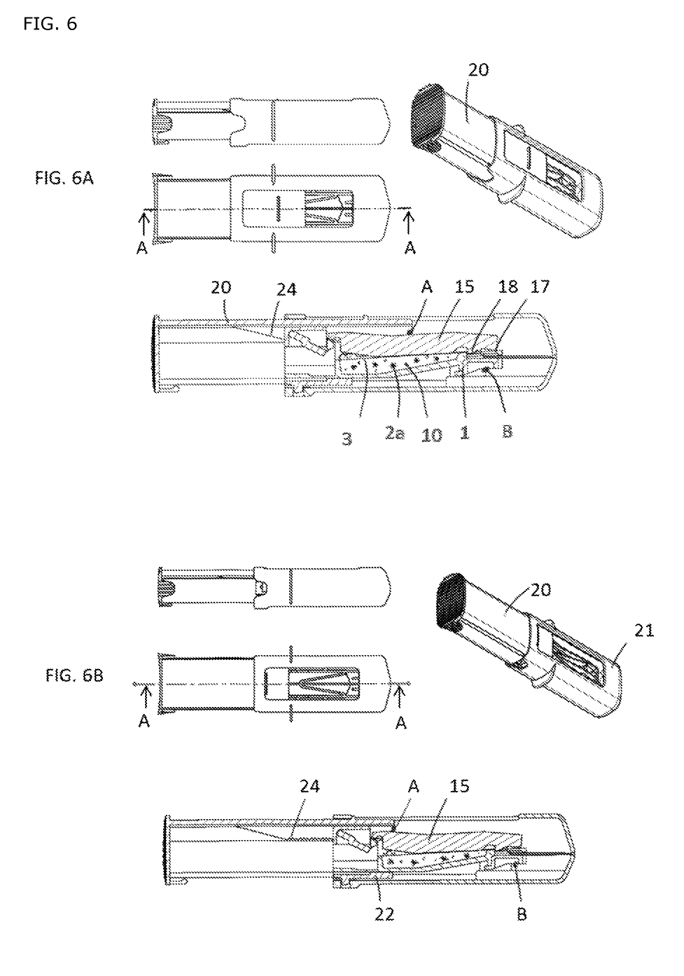

FIG. 6 shows the container of FIG. 5 in use.

FIG. 6A shows the container where the plunger 20 is partially pulled out. The lid 15 is hinged in 17 on the rigid, first outer wall 1 of the first chamber. When the plunger 20 is being pulled out, the inclined slope 24 pushes the lid 15 down, so that the protuberance 18 breaks the separation wall 3, and the atmospheric pressure acting via the flexible, second outer wall 9 of the second chamber, pushes the liquid solvent 10 into the first chamber that was under vacuum, where the liquid solvent 10 and the active substance 2a contained in the first chamber are mixed to form a liquid composition. The spring (not shown) is now stretched from point A on the plunger 20 to point B on the first, rigid outer wall 1 of the first chamber.

FIG. 6B shows the plunger 20 completely pulled out of the housing 21. When the plunger 20 is being pulled out, the inclined slope 24 vibrates the lid 15 whereby further mixing of the liquid composition is achieved. The spring (not shown), which is now stretched from point A on the plunger 20 to point B on the rigid, outer wall of the first chamber, is caught by a hook (not shown) on the guiding unit 22.

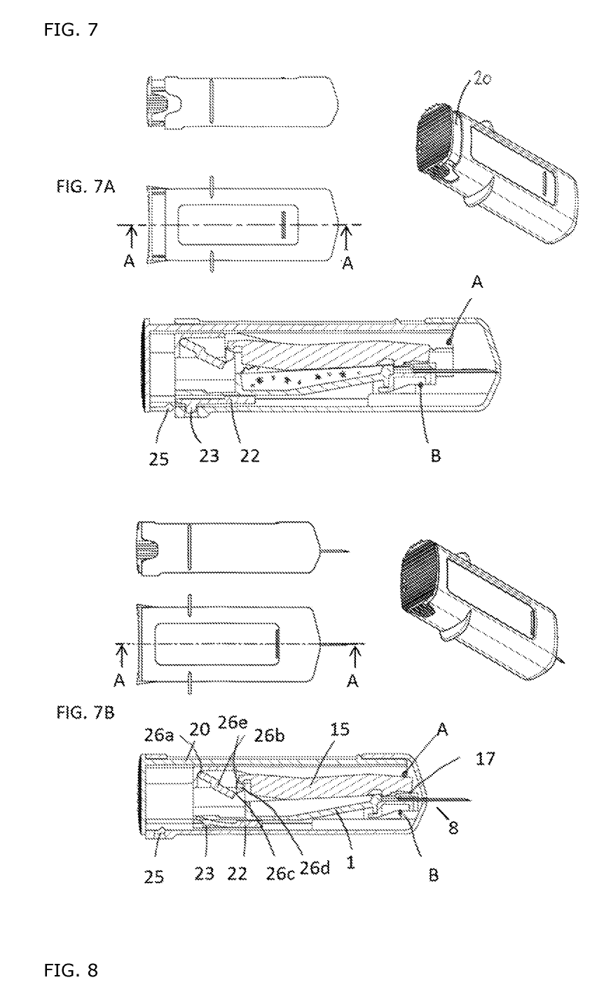

FIG. 7 shows how the needle of the container of FIG. 6 is pushed out of the injection device.

FIG. 7A shows the plunger 20 pushed partially in again until the point immediately before the spring catch 23 on the guiding unit 22 is released by the wedge 25 on the plunger 20. The spring (not shown) is now stretched from point A on the plunger 20 to the hook (not shown) on the guiding unit 22, and further to point B on the rigid, outer wall of the first chamber.

FIG. 7B shows the plunger 20 pushed completely in the device. The spring catch 23 on the guiding unit 22 is released by the wedge 25 on the plunger 20 whereby the spring power from the spring (not shown), stretched between point A on the plunger 20 to the hook (not shown) on the guiding unit 22, and further to point B on the rigid, outer wall 1 of the first chamber, has pushed the guiding unit 22 forward. The guiding unit 22 pushes a joint mechanism 26 consisting of: a hinge 26a on a connecting rod 26b, a hinge 26c for a joint 26d, and a hinge 26e on the lid 15. The lid 15 applies a pressure on the rigid outer wall 1 of the first chamber along the axis 17 so that the spring power from the spring (not shown), stretched between the guiding unit 22 and point B on the rigid outer wall of the first chamber 1, is not weakened. This results in the cannula 8, which is mounted on the rigid outer wall of the first chamber, being pushed out of the housing, and in the patient.

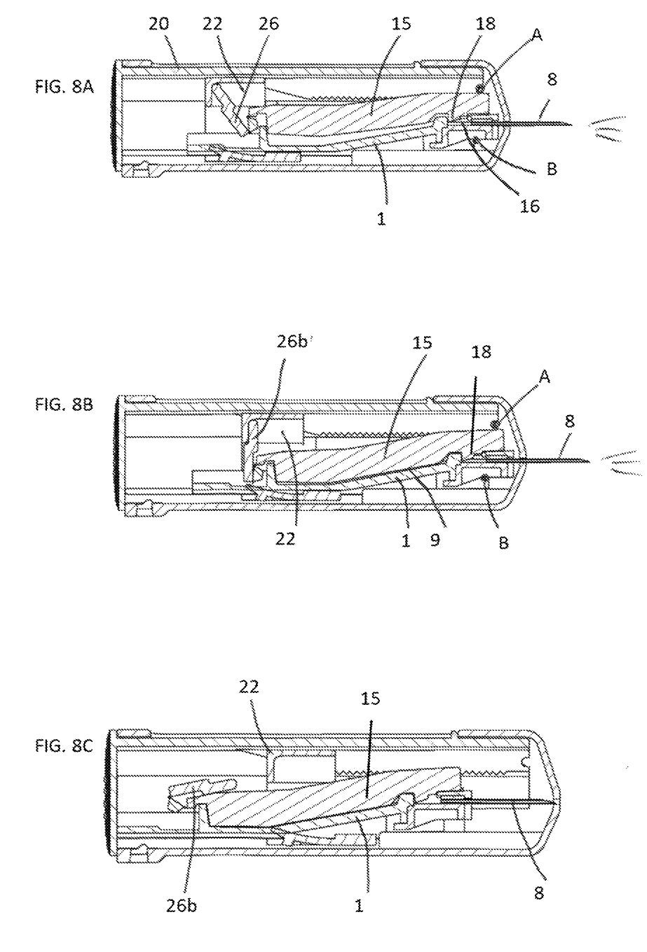

FIG. 8 shows how the liquid composition is expelled from the container and how the needle retracts within the device.

FIG. 8A. The guiding unit 22 breaks the breakable wall 16 by the action of the spring power from the spring (not shown) being stretched between point A on the plunger 20 and the guiding unit 22, together with the spring power from the spring being stretched between the guiding unit 22 and point B on the rigid outer wall 1 of the first chamber. These forces pull the guiding unit 22 forward, and the guiding unit 22 pushes the lid 15 and the protuberance 18 via the joint mechanism 26 through the breakable wall 16 so that the lid 15 expels the mixed liquid composition through the cannula 8.

FIG. 8B shows the device at the time where the container is emptied. The lid 15 has pushed the flexible, second outer wall 9 of the second chamber completely into the rigid outer wall 1 of the first chamber. At the same time, the connecting rod 26b is now perpendicular to the axis of the cannula 8.

FIG. 8C shows the injection device in the end position. The connecting rod 26b tilts over backwards, allowing the lid 15, the rigid outer wall 1 of the first chamber and the cannula 8 to be pulled by the spring power from the spring (not shown) being stretched between the guiding unit 22 and point B on the rigid outer wall 1 of the first chamber, so that the cannula 8 is completely contained within the housing.

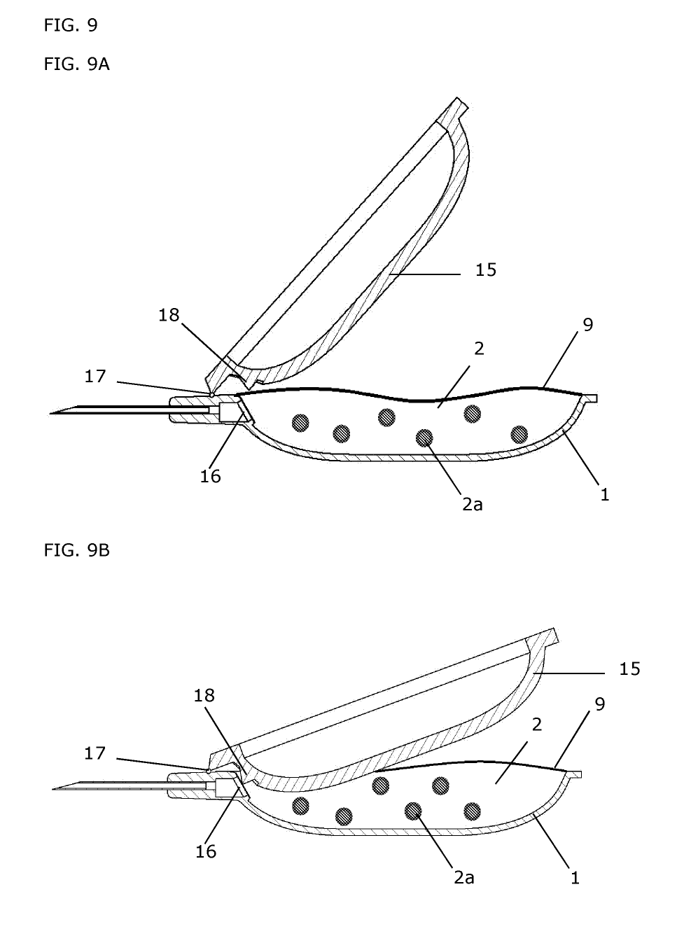

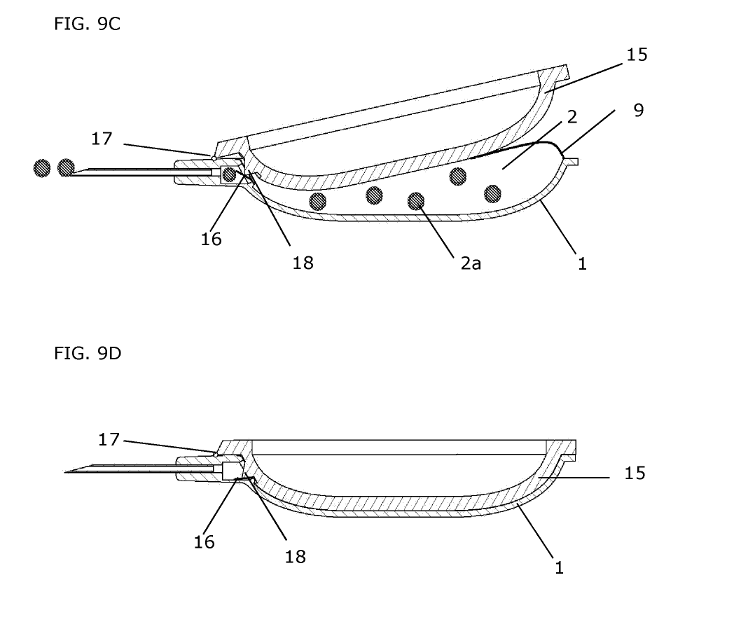

FIG. 9 shows how the liquid composition is expelled from a container with a single chamber equipped with a needle.

FIG. 9A shows a container with a single chamber in an open conformation, prior to use. The chamber is defined by a first outer wall 1 and by a flexible, second outer wall 9 and contains a liquid substance 2 comprising an active substance 2a. A moveable lid 15 is hinged to the container in 17 and comprises a protuberance 18. A breakable seal 16 prevents fluid communication between the chamber and the needle.

FIG. 9B shows the container when the moveable lid 15 is being pressed toward the chamber. The flexible wall 9 is deformed because of the pressure applied by the lid, and the protuberance 18 gets contacted with the breakable seal 16.

FIG. 9C shows the container when the moveable lid 15 is being pressed further toward the chamber. The pressure exerted on the lid causes the protuberance 18 to break the breakable seal 16, thereby allowing fluid communication between the chamber and the needle. The liquid substance 2 comprising the active substance 2a is being expelled through the needle.

FIG. 9D shows the container when the moveable lid 15 cannot be pressed further toward the chamber. The chamber is empty.

FIGS. 10-17 show a preferred embodiment which is described in detail in the below and where:

Housing 31

Housing lock 31a

Housing hole 31b

Curved path in housing 31c

Needle stop inside housing 31d

Housing feeding stop 31e

Plunger 32

Spring attachments 32a

Plunger hook 32b

Plunger teeth 32c

Plunger trigger 32d

Displacement means 33

Displacement means hooks 33a

Displacement means recess 33b

Medicine chamber 34

Medicine chamber pal 34a

Medicine chamber bottom 34c

Medicine chamber top 34e

Medicine chamber intermediate section 34g

Medicine chamber connecting rod 34i

Needle 35

Needle plastic part 35a

Needle rear end 35b

Spring or rubber band 36

Medicine bag 37

Medicine bag liquid chamber 37a

Medicine bag dry chamber 37b

Medicine bag peel section 37c

Referring to FIGS. 10a, 10b and 10c there is shown the syringe in its original position. FIG. 10a shows the syringe from the outside in perspective. FIG. 10b shows the syringe in section through the centre line. FIG. 10c shows the syringe in part, separated. The syringe comprises a housing 31, a plunger 32, a feed unit 33, a medicine chamber 34, a needle 35, a spring or rubber band 36, and a medicine bag 37. The spring or rubber band 36 is shown in FIG. 10a-c inactivated and secured on the plunger 32 in the attachment points 31a.

From there, the spring or rubber band 36 is passed around hooks 32b (only one is visible in FIG. 10c) on the plunger 32 and the needle 35. The needle 35 is thus secured. Needle 35 has a plastic portion 35a abutting the medicine chamber pal 34a whereby it is prevented from sliding backwards. Medicine chamber pal 34a is hinged to the 35 medicine chamber bottom 34c. The bottom of the medicine chamber 34c can move in the axial direction. In the bottom of the medicine chamber 34c is a medicine bag 37 attached so that it is caught between medicine chamber bottom 34c and medicine chamber top 34e which is hinged at point 34d. Medicine bag 37 consists of two chambers: the liquid chamber 37a and dry chamber 37b separated by a peel section 37c. Medicine chamber top 34e is in FIG. 10a-c in a fully open position but controlled by the plunger 32 and hinged at the point 34f to the medicine chamber via the intermediate section 34g, which is hinged at the point 34h to the medicine chamber connecting rod 34i, which in turn contacts the medicine chamber 34 in the point 34j.

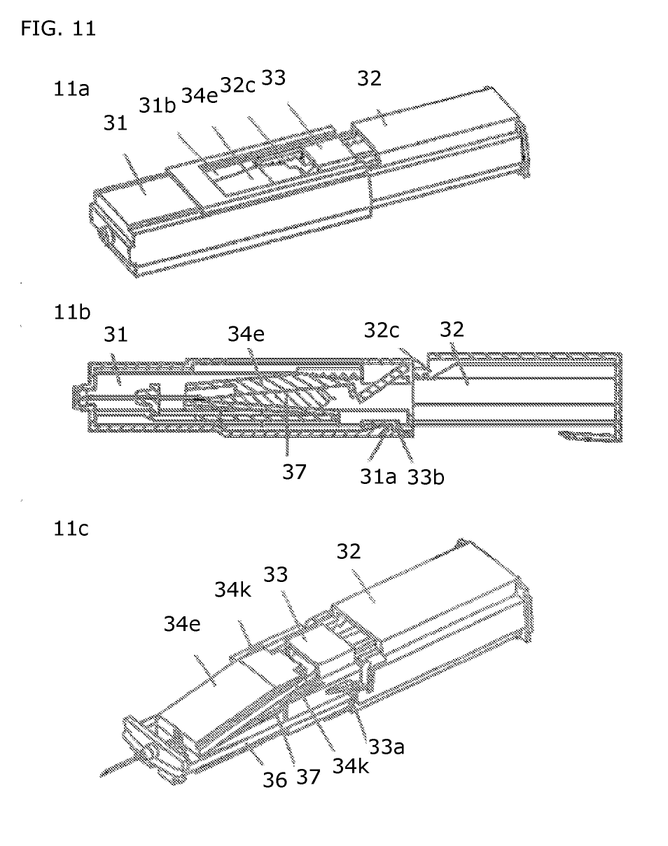

FIGS. 11a-c show the syringe with the plunger fully retracted.

FIGS. 11a show the syringe from the outside in perspective. FIG. 11b shows the syringe in section through the centre line. FIG. 11c shows the syringe in part, separated.

Upon retraction the plunger teeth 32c hits the top of the medicine chamber 34e in points 34k, whereby the medicine chamber top 34e is pressed down towards the medicine bag 37 so that the peel section 37c (see FIG. 1b) is broken and the liquid from the chamber 37a is mixed with dry matter 37b. By further retraction of the plunger 32 the plunger teeth 32c slide along the medicine chamber top 34e in points 34k, whereby the contents of the medicine bag 37 are vibrated for better mixing of liquids and solids. In the fully retracted position the spring or rubber band 36 attaches to the hooks 33a of the displacement means 33 (only one of the two are visible in FIG. 11c). Since the displacement means 33 is locked when the recess 33b rests against the housing lock 31a the plunger 32 remains in the fully open position. It is possible to inspect the medicine bag 37 through the hole 31b in the housing 31. The syringe is now ready for injection, and is placed against the skin.

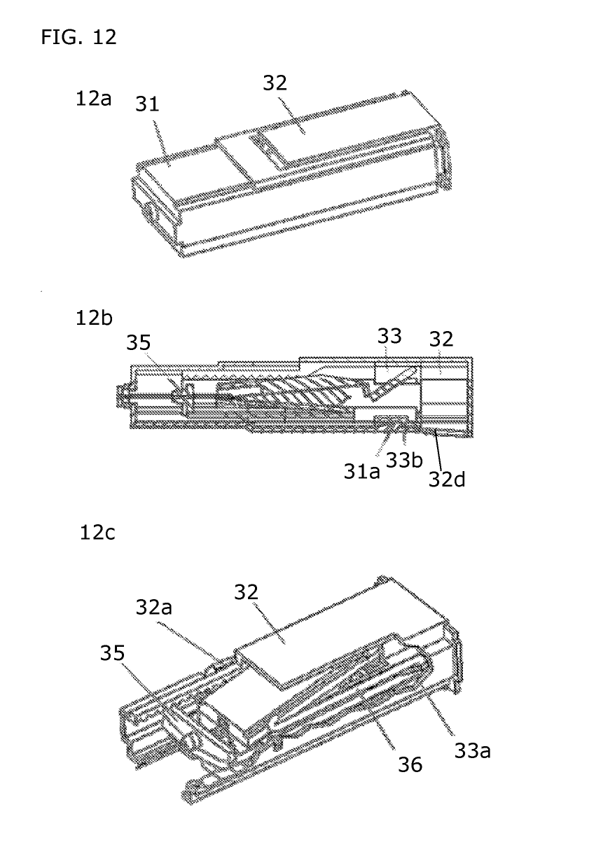

FIGS. 12a-c show the syringe with the plunger 32 pushed so that the feeding device recess 33b is almost released from the housing lock 31 by the plunger 32 triggering 32d squeezed in between them. FIG. 12a shows the syringe from the outside in perspective. FIG. 12b shows the syringe in a section through the centre line. Also in FIG. 12c the syringe partially separated. The spring or rubber band 36 is now fully activated and its course is now from the attachment points 32a to the feed device hooks 33a and towards the needle 35.

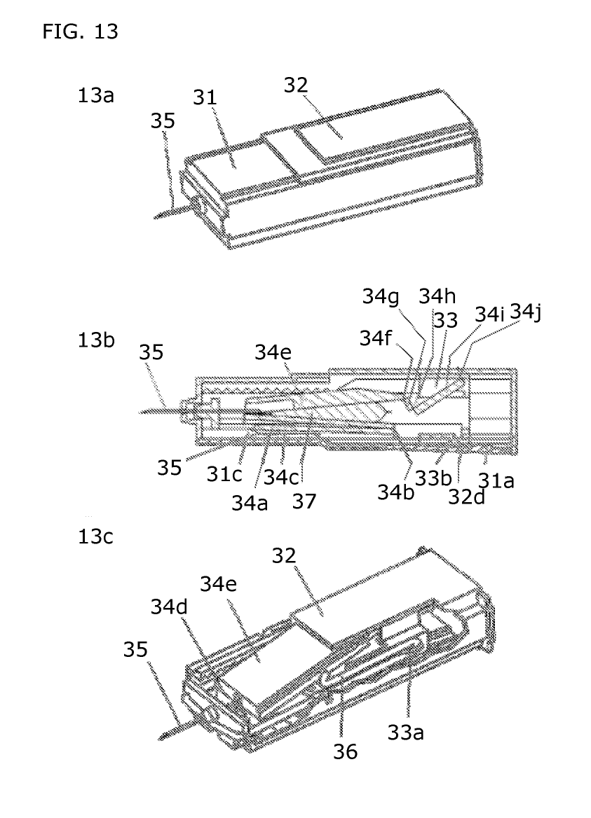

FIGS. 13a-c shows the syringe with the needle released into the skin.

FIG. 13a shows the syringe from the outside in perspective. FIG. 13b shows the syringe in section through the centre line. FIG. 13c shows the syringe in part, separated. The housing lock 31a has been released from the recess 33b by inserting the plunger trigger 32d there between. The spring or rubber band 36 pulls the displacement means 33 forward, whereby it pushes the connecting rod 34i of medicine chamber via the hinge 34j. The connecting rod 34i pushes in a forward and downward direction on the intermediate section 34g of the medicine chamber via hinge 34h. The intermediate section 34g of the medicine chamber pulls the medicine chamber top 34e in a forward and downward right direction via the hinge 34f. Medicine chamber top 34e is prevented by medicine bag 37 from moving down and hence it moves forward and pulls the medicine chamber bottom through the hinge 34d. Medicine chamber bottom 34c pushes the medicine chamber pal 34a forward via the hinge 34b. Medicine chamber pal 34a pushes the needle 35 forward to the medicine chamber pal 34a and hits the curve path 31c of the housing, whereby the medicine chamber pal 34a releases its engagement in the needle plastic portion 35a.

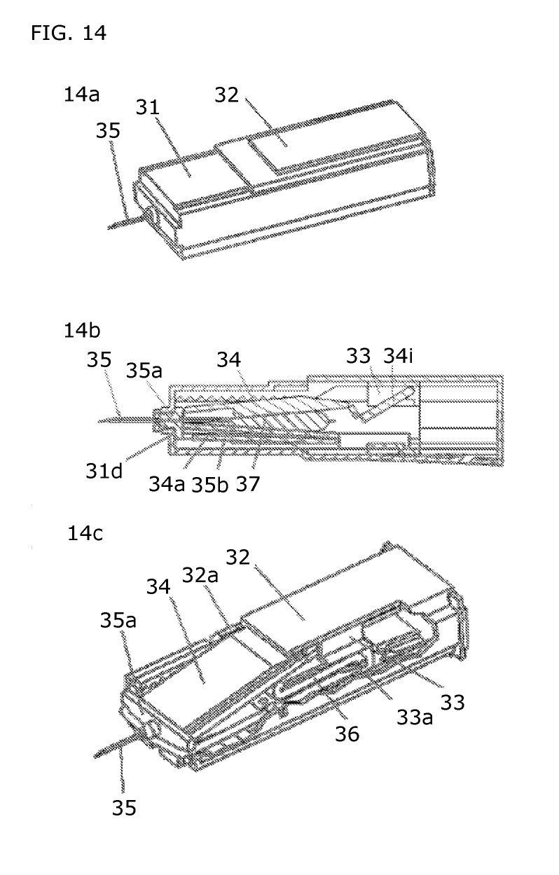

FIGS. 14a-c show the syringe with the medicine chamber completely released and ready to inject the medicine. FIG. 14a shows the syringe from the outside in perspective. FIG. 14b shows the syringe in section through the centre line, and in FIG. 14c the syringe is partially separated.

The displacement means 33 continuously pushes forward on the connecting rod 34i of the medicine chamber due to the spring force of the spring or rubber band 36 that acts between the feed device hooks 33a and the spring attachments 32a. Thereby the medicine chamber 34 continues forward and when the medicine chamber pal 34a no longer pushes the needle 35 onwards, the medicine bag frame 37 is punctured by the needle rear end 35b. The needle continues until stopped by the needle stop 31d inside the housing, while the medicine chamber continues until it hits the needle plastic part 35a.

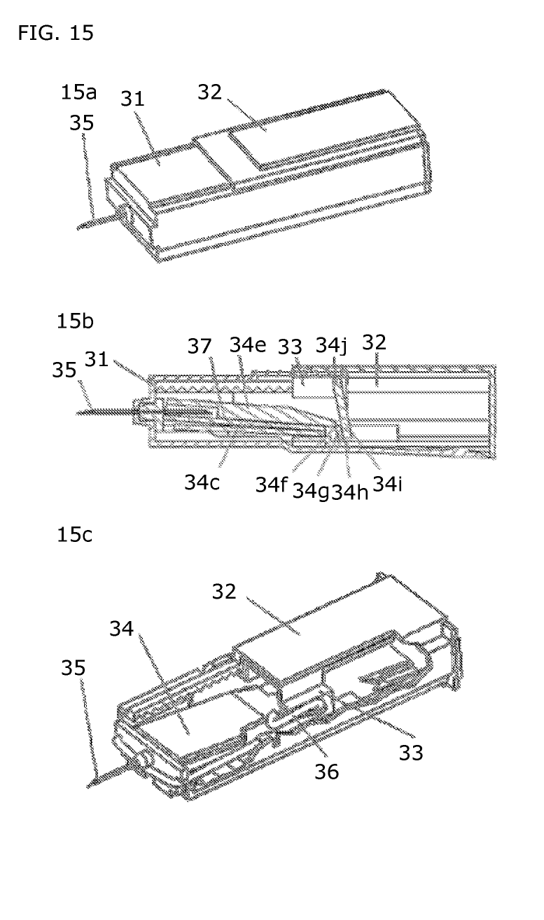

FIGS. 15a-c show the syringe when the medicine bag 37 has been emptied and the connecting rod 34i of the medicine chamber has passed. FIG. 15a shows the syringe from the outside in perspective. FIG. 15b shows the syringe in section through the center line. FIG. 15c shows the syringe partially separated.

Since the displacement means 33 continuously exerts a forward directed force from the bearing 34j on the medicine chamber connecting rod 34i, the connecting rod transforms this force to both a forward and downward force on the medicine chamber top 34e via the intermediate section 34g and the hinges 34f and 34h. As the medicine chamber 34 is prevented by the needle 35 from moving further forward the chamber top 34e is pressed down towards the medicine bag 37. The length of the medicine chamber connecting rod is adapted in such a way that when the medicine chamber connecting rod reaches the vertical position, the medicine bag 37 fully compressed, and thus emptied.

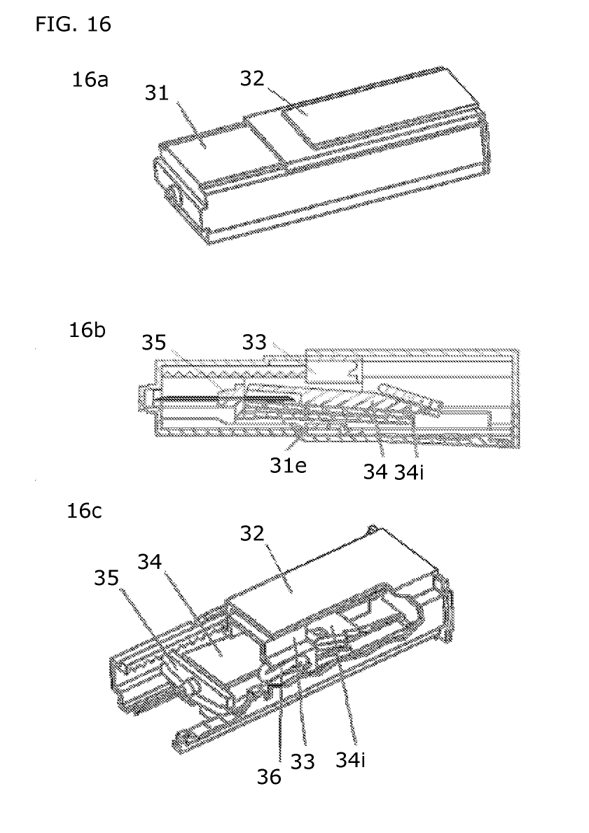

FIGS. 16a-c show the syringe in its end position.

FIG. 16a shows the syringe from the outside in perspective. FIG. 16b shows the syringe in section through the centre line. FIG. 16c shows the syringe partially separated. When medicine bag 37 is emptied the rotation of the connecting rod 34i relative to the vertical position enables the connecting rod 34i, and thus the entire medicine chamber 34, to move backward through the displacement means. The spring force of the spring or rubber band 36 that acts between the needle 35 and the displacement means pulls the needle rearward into the housing. Since the spring is longer than in the starting position it will pull the needle farther than in the original position, thus avoiding contamination risks from used syringes. The injection is now complete and the syringe can be disposed. The syringe cannot be reused since the connecting rod is no longer in engagement with the feeding device.

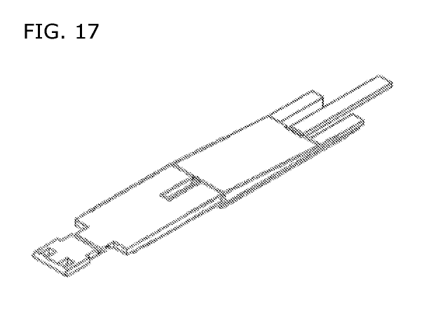

FIG. 17 shows an embodiment of the medicine chamber 34 in an open position, before assembly within the injector. In this embodiment the whole medicine chamber is manufactured from one piece.

1. A container defined by a first and a second outer walls, said container comprising:
i) at least a first closed chamber for containing an active substance, said first chamber being defined at least partially by said first outer wall;
ii) optionally a second closed chamber for containing a liquid substance comprising an active compound or a solvent, said second closed chamber being defined at least partially by said second outer wall;
wherein:
said active substance contained in the first closed chamber is in a liquid or in a solid form; and/or
said walls are arranged on each side of a middle plane; and /or
said first and second closed chambers are separated by a separation wall for separating the two closed chambers, said separation wall being impermeable and breakable upon exertion of an external force, wherein the elongation at break of the separation wall is less than the smallest of the elongations at break of the first and second outer walls; and/or
said second outer wall is adapted to be pressed towards said first outer wall for dispensing the active substance.

2. The container according to claim 1, wherein:
at least one of the first and second outer walls comprises means for facilitating breaking of said separation wall; and/or
the pressure within the second chamber is greater than the pressure within the first chamber.

3. The container according to claim 2, in which the second outer wall is more deformable than the first outer wall.

4. The container according to any of the preceding claims, in which the active substance in the first closed chamber is lyophilised.

5. The container according to claim 3, in which lyophilisation of the active substance is performed while the active substance is in the first container.

6. The container according to any of the preceding claims, in which the pressure within the first closed chamber is lower than the pressure within the second closed chamber, and the difference between the pressures in the first closed chamber and the second closed chamber is such that upon the separation wall breaking, the liquid substance of the second closed chamber flows into the first closed chamber and the substances in the first and second closed chambers are mixed.

7. The container according to any of the preceding claims, in which the active substance is a pharmaceutically active substance.

8. The container according to claim 7, in which the active substance is a vaccine.

9. The container according to any of claims 1 to 8, in which the second closed chamber contains a liquid substance comprising an active compound or a solvent.

10. The container according to claim 9, in which the second closed chamber contains a solvent.

11. The container according to claim 10, in which the solvent is such that it results in an active solution upon mixing with the active substance of the first closed chamber.

12. The container according to any of claims 1 to 11, further comprising means for attaching a needle.

13. The container according to claim 12, in which an injection needle is attached by its distal end to the means for attaching a needle, said injection needle having at least one sharp end at its proximal end.

14. The container according to claim 13, in which the distal end of the needle is a sharp end.

15. The container according to claim 14, in which the sharp distal end of the needle can break the first outer wall of the first closed chamber upon exertion of a force on the proximal end of the needle.

16. The container according to claim 12, wherein at least one of the first and second outer walls comprises means for facilitating breaking of the external wall of the first closed chamber upon exertion of a force on the first and/or the second outer walls, thereby allowing fluid communication between the first closed chamber and the distal end of the needle.

17. The container according to any of claims 12 to 14, in which the needle comprises a cap on its proximal end.

18. The container according to any of claims 1 to 17, in which the separation wall is comprised in the middle plane.

19. The container according to any of claims 1 to 18, in which the separation wall is broken upon exertion of a lateral mechanical force on the first and/or second outer walls.

20. The container according to any of claims 1 to 19, in which the means for facilitating breaking of the separation wall are protuberances on the internal sides of the first and/or second outer walls.

21. The container according to any of claims 2 to 20, in which the pressure within the first closed chamber is lower than the atmospheric pressure.

22. The container according to claim 21, in which the pressure within the second closed chamber is equal to or greater than the atmospheric pressure.

23. The container according to claim 21, in which the pressure within the second closed chamber is equal to the atmospheric pressure.

24. The container according to any of claims 1 to 21, in which the separation wall consists of a polymer selected from the group consisting of cyclic olefin copolymer and cyclic olefin polymer.

25. The container according to claim 24, in which the separation wall is rendered impermeable by treatment with paraffin or petroleum jelly.

26. The container according to any of claims 1 to 25, in which the thickness of the separation wall is comprised between 0.5 and 2.5 mm.

27. The container according to claim 26, in which the thickness of the separation wall is comprised between 1.0 and 2.0 mm, such as between 1.2 and 1.8 mm, such as between 1.4 and 1.8 mm.

28. The container according to any of claims 1 to 27, in which the first outer wall consists of metal, glass or a polymer.

29. The container according to any of claims 1 to 28, in which the first outer wall consists of a polymer selected from the group consisting of cyclic olefin copolymer and cyclic olefin polymer.

30. The container according to any of claims 1 to 29, in which the second outer wall consists of a polymer selected from the group consisting of cyclic olefin copolymer and cyclic olefin polymer.

31. The container according to any of claims 1 to 30, in which the elongation at break of the separation wall is equal to or less than 15%, such as equal to or less than 14%, such as equal to or less than 13%, such as equal to or less than 12%, such as equal to or less than 11%, such as equal to or less than 10%, such as equal to or less than 9%, such as equal to or less than 8%, such as equal to or less than 7%, such as equal to or less than 6%, such as equal to or less than 5%, such as equal to or less than 4%, such as equal to or less than 3%, such as equal to or less than 2%.

32. The container according to claim 31, in which the elongations at break of the first and second outer walls are equal to or greater than 15%, such as equal to or greater than 20%, such as equal to or greater than 30%, such as equal to or greater than 40%, such as equal to or greater than 50%, such as equal to or greater than 60%, such as equal to or greater than 70%, such as equal to or greater than 80%, such as equal to or greater than 90%, such as equal to or greater than 100%.

33. The container according to any of claims 1 to 32, in which the sum of the volumes of the first and second closed chambers is comprised between 0.1 and 5.0 mL.

34. The container according to claim 33, in which the sum of the volumes of the first and second closed chambers is comprised between 0.2 and 4.5 mL, such as between 0.3 and 4.0 mL, such as between 0.4 and 3.0 mL, such as between 0.4 and 2.0 mL, such as between 0.4 and 1.0 mL, such as between 0.4 and 0.6 mL.

35. The container according to any of claims 1 to 34, wherein the container is comprised in a housing and/or is supplied with a lid to help breaking the separation wall.

36. An injection device comprising a container according to any of claims 1 to 35 and a needle, for injecting a liquid composition.

37. The device of claim 36, in which exertion of an external force causes rupture of the separation wall, causing mixing of the substances in the first and second closed chambers to generate a liquid composition.

38. The device of any of claims 36 to 37, in which the liquid composition is an active composition.

39. The device of any of claims 36 to 38, in which the liquid composition is a vaccine composition.

40. The device of any of claims 36 to 39, in which exertion of a lateral mechanical force on the second outer wall results in collapsing of at least part of device so that the device is not reusable.

41. The device of any of claims 36 to 40, wherein the device is an automatic injection device.

42. Use of a device according to any of claims 36 to 41 for injecting a vaccine into an organism.

43. The use according to claim 42, in which the organism is a mammalian organism.

44. The use according to claim 43, in which the mammalian organism is a human organism.

45. A disposable injector comprising:
a housing having an axis extending from a distal end to a proximal end and having a proximal skin contact wall for abutting an injection region, said contact wall being provided with an aperture for receiving an injection needle;
a plunger body having a pressing surface facing in the proximal direction towards said skin contact wall and arranged for moving between a first proximal position and a first distal position, said plunger being axially displaceable within said housing between said first distal position, an intermediate position, and said first proximal position;
a medicine chamber arranged between said pressing surface and said proximal skin contact wall and axially displaceable together with said surfaces, said medicine chamber being compressible in a non-axial direction by displacing said plunger body towards said proximal end;
said needle being attached to said medicine chamber and being arranged for axial displacement between a third distal position and a third proximal position;
injection displacement means, preferably biasing means, adapted to displace said plunger body from said first distal position to said first proximal position such that said plunger body, said medicine chamber and said needle are displaced in the following sequence:
said plunger body from said first distal position to said intermediate position, and said needle from said third distal position to said third proximal position, wherein said proximal end of said needle projects distally outside said skin contact wall, and
said plunger body to said first proximal position, wherein said medicine chamber has been compressed such that at least a portion of the liquid therein has been pressed out through said proximal end of said needle,
said needle from said third proximal position to said third distal position.

46. The injector according to claim 45, further comprising a needle guide body provided with a passage for receiving and guiding said injection needle.

47. The injector according to claim 46, wherein the needle guide body has an abutment surface facing in a distal direction towards said pressing surface and arranged for axial displacement between a second distal position, a second intermediate position and a second proximal position.

48. The injector according to claim 47, wherein said injection displacement means, preferably biasing means, is adapted to displace said plunger body in said proximal direction such that said plunger body, said medicine chamber, said guide body and said needle are displaced in the following sequence:
said plunger body and said guide body from said first distal position and said second distal position, respectively, to said intermediate position and said second intermediate position, respectively, and said needle from said third distal position to said third proximal position, wherein said proximal end of said needle projects distally outside said skin contact wall, said plunger body and said guide body from said intermediate position and said second intermediate position, respectively, to said second intermediate position and said second proximal position, respectively, wherein said distal end of said needle has penetrated into said medicine chamber,
said plunger body from said second intermediate position to said first proximal position, wherein said medicine chamber has been compressed such that at least a portion of the liquid therein has been pressed out through said proximal end of said needle, and
said guide body and said needle from said second intermediate position and said third proximal position, respectively, to said second distal position and said third distal position, respectively.

49. The disposable injector according to claim 45, wherein the needle guide body is arranged so that movement of said needle guide body results in movement of the needle in the same direction.

50. The injector according to any one of the preceding claims, wherein the medicine chamber:
further contains a container for containing an injection liquid and having a proximal wall portion penetrable by said sharp distal end of said needle;
is adapted to be reduced in volume so as to compress said container to press said injection liquid into said needle.

51. The injector according to claim 50, wherein:
said plunger is capable of activating said displacement means;
said container is placed in said medicine chamber between a bottom surface and a top surface of the medicine chamber, wherein said surfaces are arranged so that a pressure can be exerted on them in a direction which is not axial, thereby providing a compressible medicine chamber movable along the axis of the injector in response to movement of the displacement means, said medicine chamber compressing the container when moved towards said proximal wall.

52. The disposable injector according to claim 51, wherein at least one of the bottom and top surfaces of the medicine chamber is connected to the plunger so that movement of the plunger results in the plane comprising said at least one surface to rotate in a non-axial direction.

53. The disposable injector according to any one of claims 51 to 52, wherein said surfaces are planar.

54. The disposable injector according to any one of claims 51 to 52, wherein said surfaces are not planar.

55. The disposable injector according to any one of claims 51 to 54, wherein the axes of said surfaces intersect at the proximal end of the injector.

56. The disposable injector according to any one of claims 52 to 56, wherein connection of the at least one surface to the plunger is achieved by the means of at least one hinge.

57. The disposable injector according to any one of claims 51 to 56, whereby a spring positioned between the injection needle and the displacement means is tensioned for being released when the plunger is retracted so that the displacement means is moved forward by the spring force.

58. The disposable injector according to any one of the preceding claims, wherein the injection displacement means comprises a rubber band.

59. The disposable injector according to any one of the preceding claims, wherein said spring is made of rubber.

60. The disposable injector according to any one of the preceding claims, wherein said container is a bag made of a plastic film.

61. The disposable injector according to any one of the preceding claims, wherein the region of the container, which is to be penetrated by said sharp distal needle end is provided with a sealing film, for instance made of rubber, which is intended to seal against the side surface of said needle, when said needle has penetrated into said bag.

62. The disposable injector according to any one of the preceding claims, wherein said container consists of:
a first chamber containing a dry matter;
a second chamber containing a liquid;
a peel section separating the first and the second chamber;
wherein the peel section is breakable.

63. The injector according to claim 62, wherein breakage of the peel section occurs upon retraction of the plunger, whereby the dry matter contained in the first chamber and the liquid contained in the second chamber are contacted, thus resulting in a liquid composition.

64. The injector according to any one of claim 62 or 63, wherein the dry matter comprises an active substance.

65. The injector according to claim 64, wherein the active substance is a pharmaceutically active substance.

66. The injector according to claim 65, wherein the pharmaceutically active substance is a vaccine.

67. The injector according to any one of claims 62 to 66, wherein the liquid contained in the second chamber comprises an active compound or a solvent.

68. The injector according to claim 67, wherein the liquid contained in the second chamber is a solvent.

69. The injector according to claim 68, wherein the solvent is such that it results in an active solution upon mixing with the active substance of the first chamber.

70. The injector according to any one of claims 62 to 69, wherein further retraction of the plunger results in vibrations allowing for mixing of the dry matter and of the liquid.

71. The injector according to any one of the preceding claims, wherein the proximal skin contact wall of the housing is provided with an antiseptic means intended for being applied to the injection site.

72. The injector according to any one of the preceding claims, wherein a cross-section of the housing is rectangular.

73. The injector according to any one of the preceding claims, wherein the housing is equipped with grips to facilitate manipulation.

74. The injector according to any one of the preceding claims, wherein the housing is equipped with a window allowing visualisation of the medicine chamber.
